(19) United States
(12) Patent Application Publication
(10) Pub. No.: US 2011/0071413 A1
(43) Pub. Date: Mar. 24, 2011

(54) MORPHOLOGY BASED ISCHEMIA DETECTION USING INTRACARDIAC ELECTROGRAMS

(76) Inventors: Yi Zhang, Plymouth, MN (US); Shibaji Shome, Minneapolis, MN (US); Kritika Gupta, Durham, NC (US); Dan Li, Shoreview, MN (US); Julie A. Thompson, Circle Pines, MN (US)

(21) Appl. No.: 12/888,900

(22) Filed: Sep. 23, 2010

Related U.S. Application Data
(60) Provisional application No. 61/245,572, filed on Sep. 24, 2009.
(60) Provisional application No. 61/302,668, filed on Feb. 9, 2010.

(51) Int. Cl. A61B 5/0452; A61N 1/365

(52) U.S. Cl. 600/509; 607/17

(57) ABSTRACT

An apparatus comprises an ambulatory cardiac signal sensing circuit configured to provide an electrical cardiac signal representative of cardiac activity of a subject and processor. The processor includes a feature module, a correlation module, and an ischemia detection module. The feature module is configured to identify a fiducial feature in the cardiac signal and locate one or more cardiac features in the cardiac signal using the fiducial feature. The correlation module is configured to calculate a measure of similarity of morphology for a segment of the cardiac signal that includes the cardiac features. The ischemia detection module is configured to detect a change in the measure of similarity and determine whether the detected change in the measure of similarity is indicative of ischemia.

### CROSS-REFERENCE TO RELATED APPLICATIONS

This application claims the benefit of U.S. Provisional Application No. 61/245,572, filed on Sep. 24, 2009, under 35 U.S.C. §119(e), which is incorporated herein by reference in its entirety.

This application also claims the benefit of U.S. Provisional Application No. 61/302,668, filed on Feb. 9, 2010, under 35 U.S.C. §119(e), which is incorporated herein by reference in its entirety.

### BACKGROUND

Implantable medical devices (IMDs) include devices designed to be implanted into a patient. Some examples of these devices include cardiac function management (CFM) devices such as implantable pacemakers, implantable cardioverter defibrillators (ICDs), cardiac resynchronization therapy devices (CRTs), and devices that include a combination of such capabilities, such as for monitoring or therapy. The devices can be used to treat patients or subjects using electrical or other therapy or to aid a physician or caregiver in patient diagnosis through internal monitoring of a patient's condition. The devices may include one or more electrodes in communication with one or more sense amplifiers to monitor electrical heart activity within a patient, and often include one or more sensors to monitor one or more other internal patient parameters. Other examples of IMDs include implantable diagnostic devices, implantable drug delivery systems, or implantable devices with neural stimulation capability.

Some IMDs include one or more sensors to monitor different aspects of the patient's cardiovascular system. Ischemia occurs when blood flow to cardiac muscles decreases below the metabolic requirements of the heart. Detecting ischemia early is critical to the health of the patient and allows early initiation of treatment. Cardiac muscle cells that are ischemic are electrically irritable and may be more susceptible to abnormal heart rhythms (e.g., fibrillation). Further, ischemia impairs the pumping function of the heart. If left untreated the underlying cause of ischemia which is commonly artherosclerotic disease may lead to myocardial infarction (i.e., heart attack).

### Overview

This document relates generally to systems, devices, and methods for detecting an ischemic event in a patient or subject. In particular, a device analyzes the morphology of sensed electrical cardiac signals in order to detect ischemia.

Example 1 includes an apparatus comprising an ambulatory cardiac signal sensing circuit configured to provide an electrical cardiac signal representative of cardiac activity of a subject and a processor communicatively coupled to the ambulatory cardiac signal sensing circuit. The processor includes a feature module, a correlation module, and an ischemia detection module. The feature module is configured to identify a fiducial feature in the cardiac signal and locate one or more cardiac features in the cardiac signal using the fiducial feature. The correlation module is configured to calculate a measure of similarity of morphology for a segment of the cardiac signal that includes the cardiac features. The ischemia detection module is configured to detect a change in the measure of similarity and determine whether the detected change in the measure of similarity is indicative of ischemia and provide an indication of ischemia to a user or process according to the detected change in the measure of similarity.

In Example 2, the ambulatory cardiac signal sensing circuit of Example 1 optionally includes a first sensing channel configured to provide a first cardiac signal and a second sensing channel configured to provide a second sensing channel. The feature module is optionally configured to identify the fiducial feature using the first cardiac signal and locate the cardiac features using the second cardiac signal. The correlation module is optionally configured to calculate the measure of similarity using a segment of the second cardiac signal.

In Example 3, the feature module of any of examples 1 and 2 is optionally configured to locate the cardiac features in the cardiac signal using SPs established in a template segment, wherein each SP corresponds to a turn encountered in the template segment. The correlation module is optionally configured to calculate a correlation coefficient (CC) that indicates a degree of similarity between a shape of the segment of the cardiac signal that includes the located SP features and a shape of the template segment.

In Example 4, the feature module of any one of Examples 1-3 is optionally configured to establish significant points (SPs) in a segment of the cardiac signal, wherein each SP corresponds to a turn encountered in the cardiac signal. The correlation module is optionally configured to calculate a measure of similarity between the established SPs of the cardiac signal segment and the established SPs of the template signal, and the ischemia detection module is optionally configured to detect a change in the measure of similarity of the established SPs.

In Example 5, the correlation module of Example 4 is optionally configured to determine the measure of similarity at least one of a number of SPs established for the cardiac signal segment, a location of a turn corresponding to an SP of the cardiac signal segment, a degree of turn of the cardiac signal segment corresponding to an SP, and an amplitude of the cardiac signal segment at a corresponding turn to an SP.

In Example 6, the feature module of any one of Examples 1-5 is optionally configured to locate one or more of a maximum value of the cardiac signal, a minimum value of the cardiac signal, a maximum slope of the cardiac signal, a peak amplitude of a T-wave in the cardiac signal, an end of a T-wave in the cardiac signal, an S-wave to T-wave segment in the cardiac signal, and a significant point in the cardiac signal.

In Example 7, the correlation module of any one of claims 1-6 is optionally configured to align the fiducial feature of the of the cardiac signal segment with a corresponding feature in a template segment of the cardiac signal and calculate a correlation coefficient (CC) that indicates a degree of similarity between a shape of the segment of the cardiac signal that includes the located cardiac features and a shape of the template segment. The ischemia detection module is optionally configured to detect a change in the CC for the segment of the cardiac signal and determine whether the change is indicative of ischemia.

In Example 8, the apparatus of Example 7 optionally includes a therapy circuit communicatively coupled to the processor and configured to provide electrical pacing therapy to the subject. The ambulatory cardiac signal sensing circuit is optionally configured to provide an electrical cardiac signal representative of cardiac depolarization. The correlation module is optionally configured to calculate the CC using a first template segment when the cardiac depolarization is representative of an intrinsic beat and calculate the CC using a second template segment when the cardiac depolarization is representative of a paced beat.

In Example 9, the ambulatory cardiac signal sensing circuit of any one of Examples 1-8 is optionally configured to sense a cardiac signal that includes a first cardiac signal segment and a second cardiac signal segment. The feature module is optionally configured to identify a fiducial feature in the first cardiac signal segment and in the second cardiac signal segment, and the correlation module is optionally configured to calculate a first measure of similarity for the first signal segment and calculate a second measure of similarity for the second segment.

In Example 10, the first cardiac signal segment of Example 9 optionally includes depolarization, the second cardiac segment optionally includes repolarization, and the feature module is optionally configured to identify the depolarization and repolarization. The correlation module is optionally configured to calculate a first measure of similarity for the first signal segment to a first template that includes depolarization and calculate a second measure of similarity for the second signal segment to a second template that includes repolarization.

In Example 11, the ischemia detection module of any one of Examples 1-10 is optionally configured to calculate variation in the measure of similarity and deem that the change is indicative of ischemia when the calculated variation in the measure of similarity exceeds a specified threshold variation value.

Example 12 includes a method comprising sensing at least one cardiac signal representative of cardiac activity of a subject using an ambulatory medical device (IMD), identifying a fiducial feature in the cardiac signal, locating one or more cardiac features in the cardiac signal using the fiducial feature, calculating a measure of similarity of morphology of a segment of the cardiac signal that includes the located cardiac features, detecting a change in the calculated measure of similarity, and determining whether the detected change in the calculated measure of similarity is indicative of ischemia and providing an indication of ischemia to a user or process according to the detected change.

In Example 13, the sensing at least one cardiac signal of Example 12 optionally includes sensing a plurality of cardiac signals, including sensing a first cardiac signal using a first sensing channel and sensing a second cardiac signal using a second sensing channel. The fiducial feature is optionally identified using the first cardiac signal, the cardiac features are optionally located in the second cardiac signal, and the measure of similarity is optionally calculated using a segment of the second cardiac signal.

In Example 14, the locating cardiac features of any one of Examples 12 and 13 optionally includes establishing significant points (SPs) in a segment of the cardiac signal, wherein each SP corresponds to a turn encountered in the cardiac signal. The calculating a measure of similarity of morphology optionally includes determining similarity between the SPs in the cardiac signal segment and SPs in a template of the signal segment, and the detecting a change in the measure of similarity optionally includes detecting a change in the established SPs.

In Example 15, the locating cardiac features of any one of Examples 12-14 optionally includes establishing significant points (SPs) in a segment of the cardiac signal, wherein each SP corresponds to a turn encountered in the cardiac signal, and the calculating a measure of similarity of morphology optionally includes calculating a correlation coefficient (CC) that indicates a degree of similarity between a shape of a segment of the cardiac signal that includes the established SPs and a shape of the template segment.

In Example 16, the locating cardiac features in the cardiac signal of any one of Examples 12-15 optionally includes locating one or more of a maximum value of the cardiac signal, a minimum value of the cardiac signal, a maximum slope of the cardiac signal, a peak amplitude of a T-wave in the cardiac signal, an end of a T-wave in the cardiac signal, an S-wave to T-wave segment in the cardiac signal, and a significant point in the cardiac signal.

In Example 17, the calculating a measure of similarity of morphology of any one of Examples 12-16 optionally includes aligning the fiducial feature of the of the cardiac signal segment with a corresponding feature in a template segment of the cardiac signal and calculating a correlation coefficient (CC) that indicates a degree of similarity between a shape of the segment of the cardiac signal that includes the feature and a shape of the template segment. The detecting a change in the calculated measure of similarity optionally includes detecting a change in the CC that exceeds a CC threshold change value.

In Example 18, the calculating the CC of any one of Examples 15 and 17 optionally includes determining at least one of patient heart rate and depolarization interval, selecting a template segment from a plurality of template segments according to the determined rate or interval, wherein the plurality of template segments correspond to different ranges of rate or interval, and determining the CC using the selected template segment.

In Example 19, the determining whether the change in the calculated measure of similarity is indicative of ischemia of any one of examples 12-18 optionally includes calculating a central tendency of the calculated measure of similarity of morphology and deeming that the change is indicative of ischemia when the calculated central tendency satisfies a specified threshold central tendency value.

In Example 20, the determining whether the change in the calculated measure of similarity is indicative of ischemia of any one of Examples 12-19 optionally includes trending the change in the calculated measure of similarity of morphology and deeming whether the change is indicative of ischemia using the trended calculated measure of similarity.

These Examples can be combined in any permutation or combination. This section is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of an example of portions of a system that includes an IMD.

FIGS. 2A and 2B show examples of sensed electrograms.

FIG. 3 is a flow diagram of an example of a method of detecting ischemia.

FIG. 4 is a block diagram of portions of an example of a device for detecting ischemia.

FIG. 5 shows a conceptualized cardiac signal segment and a template signal segment.

FIGS. 6A and 6B show conceptualized cardiac signal segments representing a cardiac signal sensed using a shock channel and a rate channel.

FIG. 7 shows a boxplot of calculated correlation coefficient values versus heart beat.

FIG. 8 shows box plots for a two-windowed approach to ischemia detection.

FIG. 9 shows a representation of a curvature signal calculated for a sensed cardiac signal segment.

### DETAILED DESCRIPTION

An IMD or other ambulatory medical device may include one or more of the features, structures, methods, or combinations thereof described herein. For example, a cardiac monitor or a cardiac stimulator may be implemented to include one or more of the advantageous features or processes described below. It is intended that such a monitor, stimulator, or other implantable or partially implantable or other ambulatory (e.g., wearable) device need not include all of the features described herein, but may be implemented to include selected features that provide for unique structures or functionality. Such a device may be implemented to provide a variety of therapeutic or diagnostic functions. Although the present document focuses on an implantable arrangement, for illustrative clarity, it is understood that in certain examples an external ambulatory (e.g., wearable) embodiment can also be provided, such as for using the subject matter described herein.

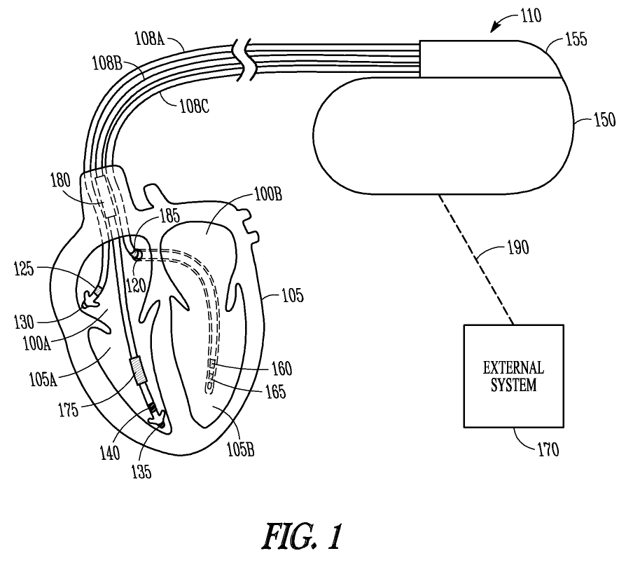

FIG. 1 is an illustration of portions of a system that uses an IMD 110. Examples of IMD 110 include, without limitation, a pacer, a defibrillator, a cardiac resynchronization therapy (CRT) device, or a combination of such devices. The system also typically includes an IMD programmer or other external device 170 that communicates wireless signals 190 with the IMD 110, such as by using radio frequency (RF) or other telemetry signals. In some examples, the external device 170 communicates with a remote system via a network. The network can be a communication network, such as a phone network or a computer network (e.g., the internet).

The IMD 110 is coupled by one or more leads 108A-C to heart 110. Cardiac leads 108A-C include a proximal end that is coupled to IMD 110 and a distal end, coupled by electrical contacts or “electrodes” to one or more portions of a heart 105. The electrodes typically deliver cardioversion, defibrillation, pacing, or resynchronization therapy, or combinations thereof to at least one chamber of the heart 105. The electrodes may be electrically coupled to sense amplifiers to sense electrical cardiac signals.

Heart 105 includes a right atrium 100A, a left atrium 100B, a right ventricle 105A, a left ventricle 105B, and a coronary sinus 120 extending from right atrium 100A. Right atrial (RA) lead 108A includes electrodes (electrical contacts, such as ring electrode 125 and tip electrode 130) disposed in an atrium 100A of heart 105 for sensing signals, or delivering pacing therapy, or both, to the atrium 100A.

Right ventricular (RV) lead 108B includes one or more electrodes, such as tip electrode 135 and ring electrode 140, for sensing signals, delivering pacing therapy, or both sensing signals and delivering pacing therapy. Lead 108B optionally also includes additional electrodes, such as for delivering atrial cardioversion, atrial defibrillation, ventricular cardioversion, ventricular defibrillation, or combinations thereof to heart 105. Such electrodes typically have larger surface areas than pacing electrodes in order to handle the larger energies involved in defibrillation. Lead 108B optionally provides resynchronization therapy to the heart 105. Resynchronization therapy is typically delivered to the ventricles in order to better synchronize the timing of depolarizations between ventricles.

The IMD 110 may include a third cardiac lead 108C attached to the IMD 110 through the header 155. The third cardiac lead 108C includes ring electrodes 160 and 165 placed in a coronary vein lying epicardially on the left ventricle (LV) 105B via the coronary vein. The third cardiac lead 108C may include a ring electrode 185 positioned near the coronary sinus (CS) 120.

Lead 108B may include a first defibrillation coil electrode 175 located proximal to tip and ring electrodes 135, 140 for placement in a right ventricle, and a second defibrillation coil electrode 180 located proximal to the first defibrillation coil 175, tip electrode 135, and ring electrode 140 for placement in the superior vena cava (SVC). In some examples, high-energy shock therapy is delivered from the first or RV coil 175 to the second or SVC coil 180. In some examples, the SVC coil 180 is electrically tied to an electrode formed on the hermetically-sealed IMD housing or can 150. This improves defibrillation by delivering current from the RV coil 175 more uniformly over the ventricular myocardium. In some examples, the therapy is delivered from the RV coil 175 only to the electrode formed on the IMD can 150.

Note that although a specific arrangement of leads and electrodes are shown the illustration, the present methods and systems will work in a variety of configurations and with a variety of electrodes. Other forms of electrodes include meshes and patches which may be applied to portions of heart 105 or which may be implanted in other areas of the body to help “steer” electrical currents produced by IMD 110.

An IMD may be configured with a variety of electrode arrangements, including transvenous, endocardial, and epicardial electrodes (i.e., intrathoracic electrodes), and/or subcutaneous, non-intrathoracic electrodes, including can, header, and indifferent electrodes, and subcutaneous array or lead electrodes (i.e., non-intrathoracic electrodes).

Monitoring of electrical signals related to cardiac activity may provide early, if not immediate, diagnosis of ischemia. An electrogram or egram is an electrical cardiac signal sensed using implantable or other ambulatory electrodes such as those described previously herein. Egrams may be sensed using electrodes to deliver electrical pacing therapy, which is sometimes called a rate channel (e.g., electrodes 140 and 135 in FIG. 1). Egrams may also be sensed using electrodes to deliver higher energy shock therapy such as cardioversion or defibrillation shock therapy, which is sometimes called a shock channel (e.g., electrode 180 and an electrode formed on IMD can 150).

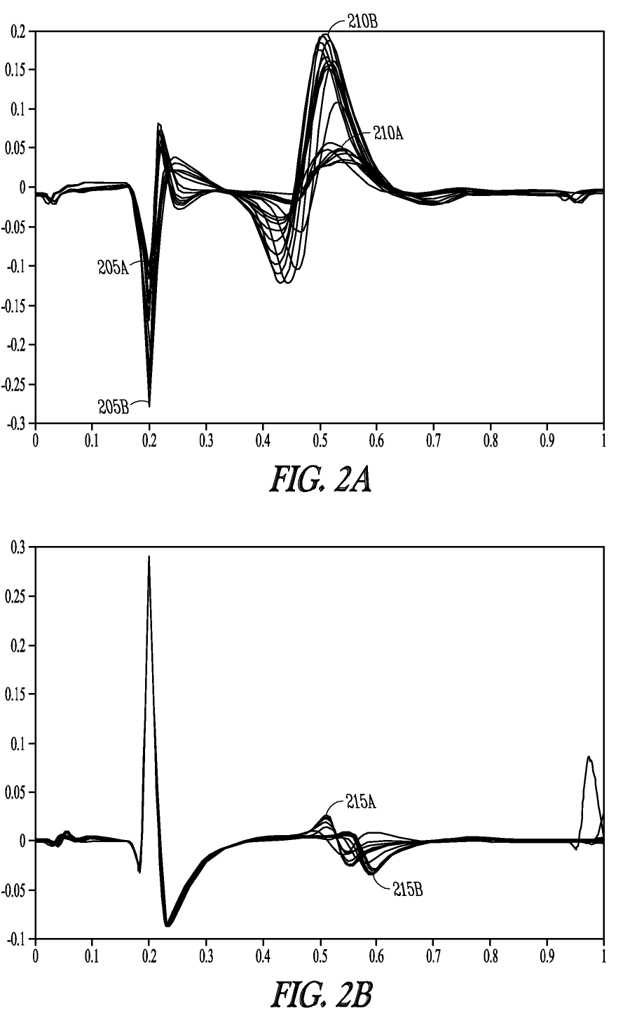

FIGS. 2A and 2B show examples of egrams sensed from swine. The egrams were sensed using a shock channel that included an RV coil electrode and a can electrode. Because of the arrangement of the electrodes involved, a cardiac signal sensed with a shock channel may provide more morphological information than a cardiac signal sensed using a rate channel. The example egrams shown include egrams (205A, 210A, 215A) that were obtained minutes before the subjects experienced an acute coronary occlusion and egrams (205B, 210B, 215B) obtained minutes after the acute coronary occlusion. The egrams demonstrate that a cardiac signal experiences morphological changes during an ischemic event. These morphological changes may thus be considered a surrogate marker of the ischemic event.

The egrams in the examples show morphological changes to the QRS complex (e.g., 205B), which represents depolarization of the ventricles. The egrams also show morphological changes to the T-wave (e.g., 210B), which represents repolarization of the ventricles. The morphological changes due to ischemia may include a deviation in the S-T interval, a change in the slope of the S-T interval, a change in one or both of amplitude and width of the T-wave, a change in one or both of the amplitude and width of the QRS complex, and a change in the timing of designated fiducial events such as a change in the QT interval. It can be seen from the egrams in the Figure that ischemic events can be detected by sensing changes in cardiac signal morphology. Ischemia detection is complicated by the fact that the changes in morphology may be different for different patients or may be different for different ischemic episodes of the same patient.

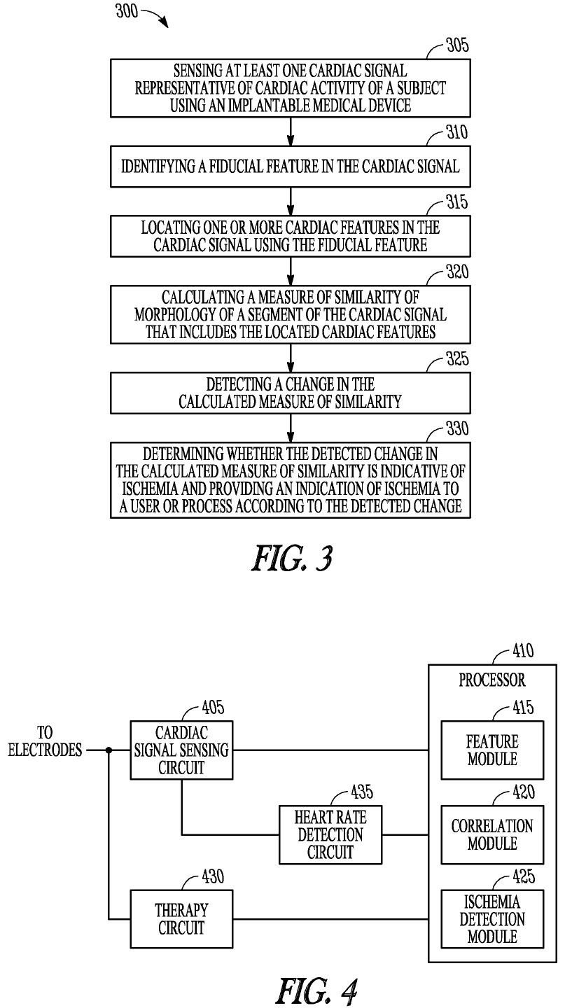

FIG. 3 is a flow diagram of an example of a method 300 of detecting ischemia. At block 305, a cardiac signal is sensed using an IMD, such as the IMD described previously in regard to FIG. 1 for example. At block 310, a fiducial feature is identified in the cardiac signal. In some examples, the fiducial feature includes an R-wave peak in the sensed cardiac signal. An R-wave refers to the first typically positive deflection in the QRS complex of an ECG.

At block 315, one or more cardiac features are located in the cardiac signal. Some examples of these cardiac features include a maximum value of the cardiac signal, a minimum value of the cardiac signal, and a maximum slope of the cardiac signal. The cardiac features may be located using the fiducial feature. For instance, if the fiducial feature is an R-wave peak, cardiac features may be located as the maximum slope of the cardiac signal preceding or following the R-wave peak or the minimum value of the cardiac signal preceding or following the R-wave peak.

Other examples, cardiac features are related to the T-wave in a cardiac signal, such as the peak amplitude of a T-wave in the cardiac signal, the slope of the T-wave (rising and/or falling), an end of a T-wave in the cardiac signal, and an S-wave to T-wave segment in the cardiac signal. The S-wave refers to a typically negative deflection in an ECG that follows the R-wave of the QRS complex. The T-wave is typically in the same direction as the R-wave. In some examples, the cardiac features related to the T-wave are located in relation to an identified R-wave peak. In some examples, the T-wave is the fiducial feature used to locate the cardiac features.

At block 320, a measure of similarity is calculated of the morphology of a segment of the cardiac signal that includes the located cardiac features. In some examples, the segment of cardiac signal is compared to a template of the signal, such as a template stored in a memory of the IMD for example. A template can be thought of as a snapshot of a cardiac signal of the subject (e.g., when the subject is ischemia-free). Similarity is measured between the sensed cardiac signal and the template of the signal. In some examples, the measure of similarity in morphology is calculated on a beat-to-beat basis. A beat-to-beat calculation provides a measure of similarity to the template from one beat to the next to quickly uncover beat-to-beat trends. In some examples, the measure of similarity in morphology is calculated on a determined central tendency of the cardiac signal. For instance, the cardiac signal may be averaged over a time window having a specified number of heart beats or a specified duration of time.

At block 325, a change in the calculated measure of similarity is detected. At block 330, it is determined whether the detected change in the calculated measure of similarity is indicative of ischemia. An indication of ischemia is provided to a user or process according to the detected change.

FIG. 4 is a block diagram of portions of an example of a device 400 for detecting ischemia. The device 400 includes an implantable or other ambulatory cardiac signal sensing circuit 405. The cardiac signal sensing circuit 405 provides an electrical cardiac signal representative of cardiac activity of a subject. In some examples, the cardiac signal sensing circuit 405 includes at least one electrode communicatively coupled to a sense amplifier circuit. In some examples, the electrode is a coil electrode such as electrodes 175, 180 in FIG. 1 or an electrode formed on the IMD can 150. In some examples, the electrode is a tip or ring electrode such as electrodes 135 and 140 in FIG. 1. In some examples, the cardiac signal sensing circuit 405 includes a sampling circuit to provide digital sampled values of the cardiac signal.

The device 400 includes a processor 410 communicatively coupled to the cardiac signal sensing circuit 405. The communicative coupling allows the cardiac signal sensing circuit 405 to communicate signals with the processor 410 even though there may be intervening circuitry. In various examples, the processor 410 includes a microprocessor, a digital signal processor, or application specific integrated circuit (ASIC). The processor 410 includes one or modules to perform the functions described. A module may include hardware, software, firmware, or any combination of hardware, software, or firmware. More than one function may be performed by a module.

The processor 410 includes feature module 415 to identify a fiducial feature in the cardiac signal and locate one or more cardiac features in the cardiac signal using the fiducial feature. Examples of fiducial features useful for identification and examples of cardiac features locatable using the fiducial feature were described previously in regard to FIG. 3.

The processor 410 also includes a correlation module 420 that calculates a measure of similarity of morphology for a segment of the cardiac signal that includes the cardiac features. In some examples, the device 400 includes a memory integral to or communicatively coupled to the processor 410 to store the template. In some examples, the processor includes a template module configured to generate one or more templates of cardiac signals sensed from subject. An approach for generating electrical cardiac signal templates using a snapshot of the subject's conducted heart beats is described in Kim et al., U.S. Pat. No. 6,708,058, entitled “Normal Cardiac Rhythm Template Generation System and Method,” filed Apr. 30, 2001, which is incorporated herein by reference in its entirety. In some examples, the measure of similarity to a template is calculated each beat and evaluated on a beat-to-beat basis to identify beat-to-beat trends. In some examples, the measure of similarity to a template is calculated on a cardiac signal that is averaged over a time window having a specified number of heart beats or a specified duration of time.

In certain examples, the measure of similarity includes a correlation coefficient (CC), or a feature correlation coefficient (FCC). The CC calculated by the correlation module 420 indicates the degree of similarity between a shape of the segment of the cardiac signal that includes the located cardiac features and a shape of the template segment. Examples of calculating correlation coefficients are discussed in U.S. Pat. No. 6,708,058, “Normal Cardiac Rhythm Template Generation System and Method,” filed Apr. 30, 2001, which is incorporated herein by reference in its entirety.

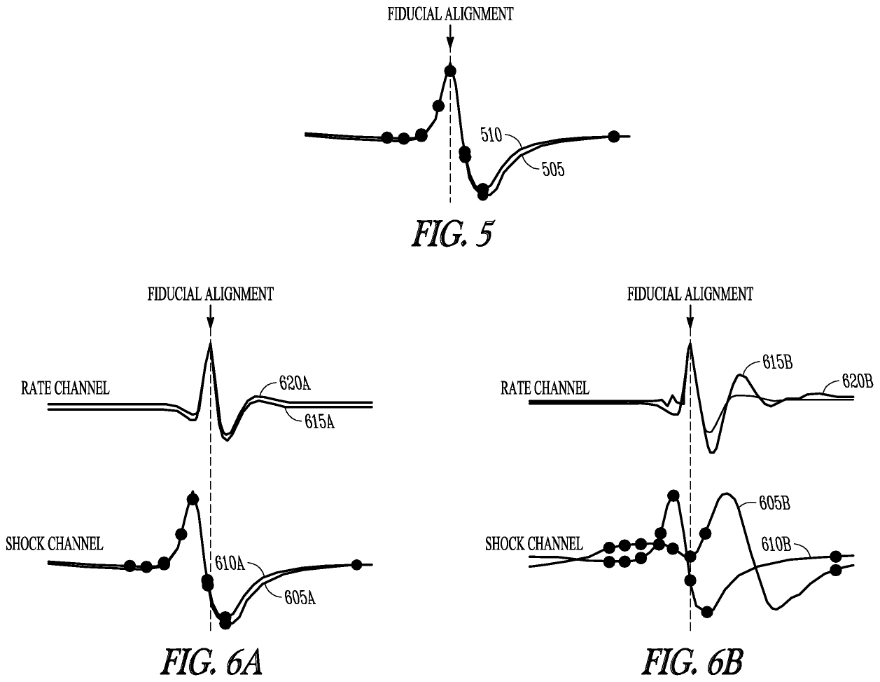

FIG. 5 shows a conceptualized cardiac signal segment 505 (i.e., not real data) and a template signal segment 510. The correlation module 420 aligns the fiducial feature of the cardiac signal segment 505 with the corresponding feature in the template segment 510. In some examples, the correlation module 420 then uses N comparison points (x₁, x₂, . . . x_{N}: y₁, y₂, . . . y_{N}) to calculate the CC. In certain examples, N=8 and the CC is calculated by

$$\frac{{( 8 {\sum }_{i = 1}^{8} {x}_{i} {y}_{i} - ( {\sum }_{i = 1}^{8} {x}_{i} ) ( {\sum }_{i - 1}^{8} {y}_{i} ) )}^{2}}{( 8 {\sum }_{i = 1}^{8} {x}_{i}^{2} - {( {\sum }_{i = 1}^{8} {x}_{i} )}^{2} ( 8 {\sum }_{i = 1}^{8} {y}_{i}^{2} - {( {\sum }_{i = 1}^{8} {y}_{i} )}^{2} )} .$$

The feature module 415 may use different signals to identify the fiducial feature and to locate the cardiac features. In some examples, the cardiac signal sensing circuit 405 includes multiple sensing channels, such as a first sensing channel configured to provide a first cardiac signal, and a second sensing channel configured to provide a second sensing channel. The feature module 415 identifies the fiducial feature using the first cardiac signal and locates the cardiac features using the second cardiac signal. The correlation module 420 is configured to calculate the measure of similarity using a segment of the second cardiac signal.

This is shown in FIGS. 6A and 6B. The signals 605A, 605B represents a signal sensed using a shock channel and the template 610A, 610B is for a shock channel comparison. The signal 615A, 615B represents a signal sensed using a rate channel and the template 620A, 620B is for a rate channel comparison. The rate channel signal is sensed in a known relationship to the shock channel signal (e.g., sensed at the same time). The correlation module 420 aligns the fiducial feature in the rate channel signal 615A, 615B with the corresponding fiducial feature in the rate channel template 620A, 620B. Because the timing relationship to the shock channel is known, the correlation module 420 is able to align shock channel signal 605A, 605B and shock channel template 610A, 610B with the fiducial feature. The correlation module 420 then calculates a measure of similarity for the shock channel. As explained previously, a cardiac signal sensed with a shock channel may provide more morphology information than a cardiac signal sensed using a rate channel.

FIG. 6A is a representation of a sampled cardiac signal segment 605A correlating well with the template. FIG. 6B is a representation where the correlation is not as good. In some examples, if the correlation is >90%, the sensed signal is deemed to correlate with the template. If the correlation ≦90%, the sensed signal is deemed to be uncorrelated. The threshold used to determine if signals are similar (e.g., if they correlate) may be programmable.

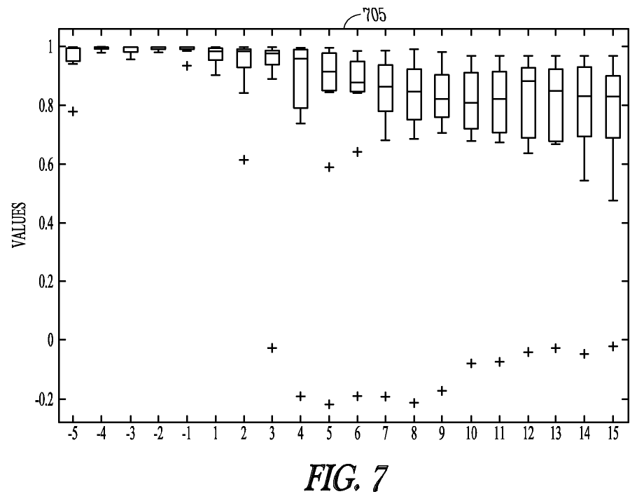

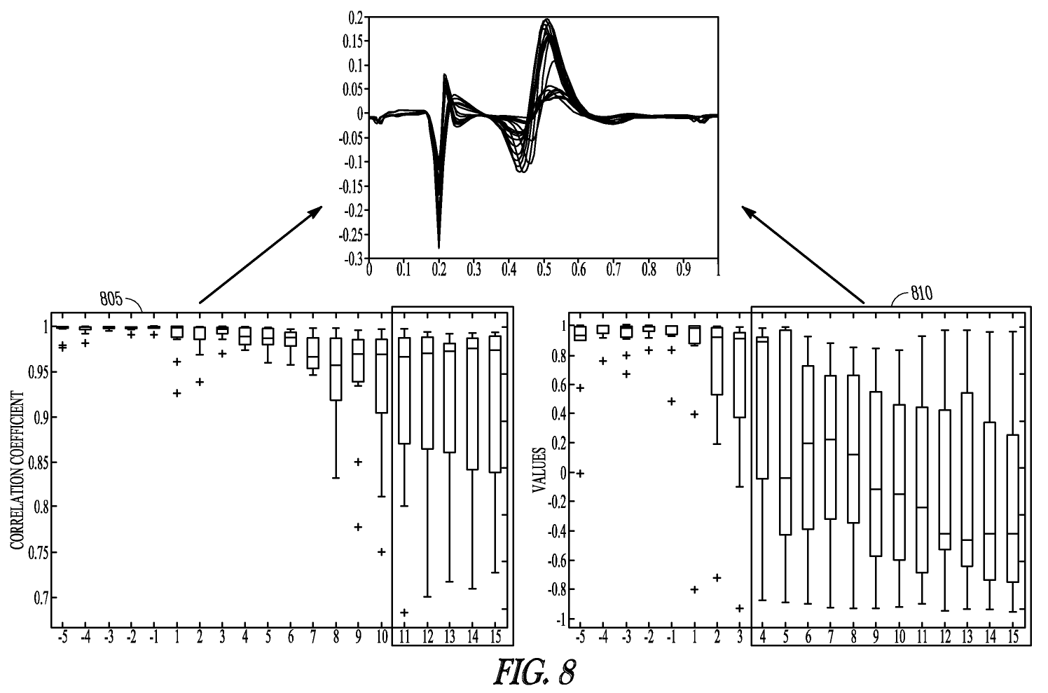

In FIG. 2A, the elevation in the S-T segment and the change in amplitude of the QRS complex due to ischemia will result in less correlation of the cardiac signal with a template and will reduce the measure of similarity. FIG. 7 is a boxplot 705 of the calculated CC values versus heart beat for episodes of acute myocardial infarction (AMI). The CC values are a measure of similarity of sensed cardiac signals to a template obtained by averaging sensed cardiac signals. The boxplot shows the variation in CC due to an occurrence of AMI in the episodes.

According to some examples, the processor 410 of FIG. 4 includes an ischemia detection module 425. The ischemia detection module 425 detects a change in the measure of similarity and determines whether the detected change in the measure of similarity is indicative of ischemia. The ischemia detection module 425 provides an indication of ischemia to a user or process according to the detected change in the measure of similarity.

In some examples, the ischemia detection module 425 determines the change in the measure of similarity is indicative of ischemia according to the time frame of the change. For instance, if the measure of similarity changes (e.g., decreases) by more than a threshold correlation value within a specified time period (e.g., ten minutes), the ischemia detection module may deem that the change is indicative of an ischemic event.

In another example, if the measure of similarity changes is determined on a beat-to-beat basis and changes by more than a threshold measurement change value within a specified number of beats (e.g., ten beats), the ischemia detection module 425 may deem that the change is indicative of an ischemic event. In yet another example, the ischemia detection module 425 tracks the value of the measure of similarity for Y heart beats (e.g., ten consecutive heart beats). If X of the Y heart beats, where X an integer less than or equal to Y (e.g., eight of ten heart beats), have a measure of similarity less than a threshold correlation value, the ischemia detection module 425 may deem that the change is indicative of an ischemic event.

In some examples, the ischemia detection module 425 calculates a central tendency (e.g., the average) of the measure of similarity over multiple beats, and deems that the change is indicative of ischemia when the calculated central tendency changes by more than a threshold central tendency change criteria. In some examples, the ischemia detection module 425 calculates variation in the measure of similarity and deems that the change is indicative of ischemia when the calculated variation in the measure of similarity exceeds a specified threshold variation value.

In some examples, the ischemia detection module 425 trends the calculated beat-to-beat measure of similarity (such as by recurrently calculating the CC and storing calculated CCs over a specified period of time). The ischemia detection module 425 deems whether the change is indicative of ischemia using the trended correlation measure.

A complication occurs when the morphology of cardiac signals of the subject change reasons unrelated to ischemia. For instance, the device 400 may be an implantable or other ambulatory pacemaker that includes a therapy circuit 430 communicatively coupled to the processor 410 to provide electrical pacing therapy to the subject. The ambulatory cardiac signal sensing circuit 405 provides an electrical cardiac signal representative of cardiac depolarization. The cardiac depolarization may be due to an intrinsic beat or a paced beat. The morphology of an intrinsic beat often may be different from the morphology of a paced beat. In certain examples, the therapy circuit further provides high energy shock therapy to the subject.

The correlation module 420 may use different templates for the measurement of similarity, such as using a first template for an intrinsic beat and a second template for a paced beat. For instance, the correlation module 420 may calculate the CC using a first template segment when the cardiac depolarization is representative of an intrinsic beat and calculate the CC using a second template segment when the cardiac depolarization is representative of a paced beat. In certain examples, loss of pacing capture is detected from a sudden appearance of low amplitude in a pacing artifact of a sensed electrogram. The correlation module 420 calculates the CC using a first template segment when the cardiac depolarization is representative of capture and calculates the CC using a second template segment when the cardiac depolarization is representative of loss of capture.

In some examples, the correlation module 420 uses different templates depending on heart rate. The device 400 may include a heart rate detection circuit 435 communicatively coupled to the cardiac signal sensing circuit 405. The heart rate detection circuit 435 may be a module integral to the processor or may be a separate circuit, such as a peak detector circuit to detect R-waves in the cardiac signal for example. The correlation module 420 calculates the CC using a first template segment when a heart rate is below a specified heart rate threshold value, and calculates the CC using a second template segment when the heart rate is above or equal to the specified heart rate threshold value. Multiple templates corresponding to different heart rate ranges (or to different depolarization interval ranges) can be stored in memory of the device 400. In some examples, the correlation module 420 uses a different template for different heart rate ranges. The template closest to the detected heart rate or interval is chosen for calculating the similarity measurement.

High or rapid heart rate may be indicative of tachyarrhythmia. Tachyarrhythmia includes ventricular tachycardia (VT) which originates from the ventricles. Tachyarrhythmia also includes rapid and irregular heart rate, or fibrillation, including ventricular fibrillation (VF). Abnormally rapid heart rate can also be due to supraventricular tachycardia (SVT). SVT is less dangerous to the patient than VT or VF. SVT includes arrhythmias such as atrial tachycardia, atrial flutter, and atrial fibrillation. A rapid heart rate can also be due to sinus tachycardia, which is a normal response to, for example, exercise or an elevated emotional state. Heart rate can be compared to one or more of a VT-1 rate zone, a VT rate zone, or a VF rate zone to detect and classify a detected tachyarrhythmia as slow VT, VT, or VF respectively. In some examples, the processor 410 suspends ischemia detection when the heart rate exceeds a lowest tachyarrhythmia detection zone. This may free up resources for morphology analysis to be used for tachyarrhythmia detection and classification.

The process of calculating a CC, such as explained in regard to FIGS. 6A and 6B, can be thought of as setting a detection window around the depolarization-repolarization event from the onset of the QRS complex to the end of the T-wave. In some examples, the detection window is centered around a repolarization event (e.g., the T-wave). In some examples, the process can include multiple detection windows arranged around different cardiac events. The correlation module 420 may calculate a measure of similarity for each of the detection windows.

In certain examples, detection is divided into a first window that includes the QRS complex associated with ventricular depolarization (and may include the P-wave associated with atrial depolarization as well) and a second window that includes the S-T to T-wave end (ST-T) segment associated with repolarization. The cardiac signal sensor is configured to sense a cardiac signal that includes a representation of the cardiac depolarization and repolarization, and the feature module 415 identifies the depolarization and the repolarization. The correlation module 420 calculates a first measure of similarity for a first signal segment that includes the depolarization and calculates a second measure of similarity for a second segment that includes the repolarization.

FIG. 8 shows box plots for the two-windowed approach to ischemia detection. The first box plot 805 shows CC values calculated for the cardiac signal segment that includes the QRS complex for the egrams of FIG. 2A, and the second box plot 810 shows CC values calculated for the ST-T segment of the cardiac signal. The CC values were calculated using a cardiac signal segment template obtained from averaging of sensed cardiac signals. The boxplots show the variation in CC values due to AMI. A comparison of FIG. 7 and FIG. 8 shows that splitting the heart beat into multiple windows may result in improved detection of AMI over one window. In certain examples, the correlation module 420 uses a fiducial feature in each of the multiple windows when calculating the CCs. In certain examples, the correlation module 420 uses multiple fiducial features in each of the multiple windows to compensate for varying heart rate.

Another method used to analyze a cardiac signal segment for detection of ischemia includes significant point (SP), or characteristic point, analysis. In a sampled cardiac signal segment, a SP corresponds to a turn encountered in the cardiac signal. Curvature for the cardiac signal segment is calculated on a sample by sample basis. In some examples, curvature is calculated according to

$$\begin{matrix}Curvature ( i \cdot Δ T ) = \frac{\frac{{d}^{2} V ( t )}{d {t}^{2}} \cdot W}{{[ 1 + {\{ \frac{d V ( t )}{d t} \cdot W \}}^{2} ]}^{3 / 2}} \\ = \frac{2 \cdot Ci \cdot W}{{[ 1 + {\{ Bi \cdot W \}}^{2} ]}^{3 / 2}} . ,\end{matrix}$$

where t=i·ΔT is the time of the sampled cardiac signal segment where curvature is calculated, W is the ratio G/U (where G is a dimensionless gain applied to the input signal and U is a constant with dimensions of voltage/time) and is selected so that a square in voltage-time space is represented by a square is sample-sample space, and Ci and Bi are coefficients obtained from minimizing error of the voltage-time sample space. SPs are established where the curvature exceeds a specified threshold curvature value.

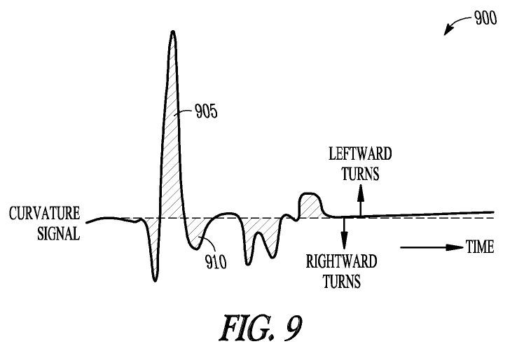

FIG. 9 shows a representation of a curvature signal 900 calculated for a sensed cardiac signal segment. The curvature signal 900 is representative of the value of calculated curvature in the cardiac signal versus time. Turns in the original cardiac signal segment are reflected as excursions above and below zero in the curvature signal 900. Each lobe of the curvature signal above zero (for example, lobe 905) or below zero (for example, lobe 910) represents a single turn in the input signal. Curvature lobes of opposite directions reflect opposite turns (leftward or rightward) in the curvature signal 900. The area under each lobe reflects the total angle included in the turn. A point-by-point process is used to identify the lobes as they occur and to find the area and centroid of each lobe. In some examples, each SP has a set of values including the time of occurrence of the SP, the amplitude of signal at that time, and a value describing the degree (e.g., the direction and extent or area) of the turn or curve in the cardiac signal that produced the SP. A description of systems and methods for morphology analysis of cardiac signals using significant points, or characteristic points, can be found in Sweeney et al., U.S. Patent Publication No. 2007/0203419 A1, filed Jun. 27, 2003, which is incorporated herein by reference in its entirety.

According to some examples, the device 400 of FIG. 4 detects ischemia using SPs. The SPs may be used as the cardiac features. The feature module 415 identifies a fiducial feature in a sensed cardiac signal segment. The feature module 415 then calculates a curvature signal for the cardiac signal. Using the curvature signal, the feature module 415 establishes one or more SPs. The feature module 415 also establishes one of more of the time of occurrence of the SP (e.g., position), the amplitude of signal at that time, and a value describing the degree of the turn or curve corresponding to the SPs in the sensed cardiac signal. Using the time of occurrence of the SPs, the position of the SPs is identified in the sensed cardiac signal.

In some examples, the correlation module 420 calculates a measure of similarity of the morphology between a segment of the sensed cardiac signal that includes the identified SPs and a template signal segment. In certain examples, the measure of similarity is a CC and the ischemia detection module 425 determines whether a detected change in the CC is indicative of ischemia. In certain examples, the measure of similarity is calculated and evaluated beat-to-beat. In some examples, the measure of similarity is calculated on a cardiac signal averaged over a time window having a specified number of heart beats or a specified duration of time.

In some examples, the measure of similarity includes a determined similarity between the SPs of the cardiac signal segment and the SPs of the template signal. The correlation module may determine one or more of the number of SPs, the position of the SPs, the amplitude of the signal at the time of an SP, and the degree of the curve in the signal at the time of the SP. The ischemia detection module 425 detects the changes in the determined similarity between the SPs of the cardiac signal segment and the SPs of the template signal. The ischemia detection module 425 then determines that the detected change in the determined similarity is indicative of ischemia when the detected change satisfies a specified change criterion. In some examples, the ischemia detection module 425 detects ischemia when the number of SPs for cardiac signal segments increases above a specified SP number threshold within a specified period of time or specified number of beats, or when a change in the number of SPs exceeds a threshold change number within a period of time or number of beats. In another example, the ischemia detection module 425 detects ischemia when the changes in the amplitude of the signal at the time of an SP exceed a specified threshold within a specified period of time.

In some examples, instead of (or in addition to) using SP analysis, the correlation module 420 calculates a CC coefficient for a curvature signal calculated for the suspected ischemic episode. This curvature CC is used to detect ischemia. For instance, the correlation module 420 calculates the curvature signal for a cardiac signal segment. The feature module 415 may then identify a fiducial feature in the curvature signal, such as by identifying the first turn to exceed a specified curvature threshold, or by identifying the turn with the largest area for example. The fiducial feature is used to align the calculated curvature segment with a template curvature signal segment. In certain examples, the curvature signal template is obtained from averaging curvature signal calculated from a set of sampled cardiac signal segments.

The correlation module 420 then calculates a CC that indicates a degree of similarity between the shape of the calculated curvature signal and the shape of a template of the curvature signal. The ischemia detection module 425 detects a change in the curvature CC. The ischemia detection module 425 deems that the change indicates ischemia when the calculated curvature CC exceeds a specified curvature CC threshold change value within a specified time period or within a specified number of heart beats.

This concept can be expanded to include any signal derived from the “raw” sensed cardiac signal. The fiducial module 415 identifies a fiducial feature in a segment of the derived signal and aligns the segment with a template of the derived signal segment. The correlation module 420 calculates a CC for the derived signal that indicates a degree of similarity between a shape of a segment of the derived signal and a shape of a template of the derived signal. The ischemia detection module 425 deems that a detected change in the derived signal CC indicates ischemia when the calculated CC satisfies a specified CC threshold change criterion within a specified time period or within a specified number of heart beats.

In some examples, the derived signal is a first derivative or slope of the sensed cardiac signal. The slope cardiac signal can be obtained by calculating the value of the difference between sampled values of the sensed cardiac signal. The slope cardiac signal may then be compared to a template of the slope signal. In some examples, the derived signal is the root mean square (RMS) of the sensed cardiac signal. The RMS cardiac signal can be obtained by squaring a specified number N of sampled values of the sensed cardiac signal, averaging the N squared values, and taking the square root of the averaged squared values. The RMS cardiac signal may then be compared to a template RMS cardiac signal. In certain examples, N is programmable integer. In certain examples, N is equal to 1. In some examples, the derived signal is the RMS of several cardiac cycles.

AMI may cause morphological changes to sensed cardiac signals that can include one or more of a deviation in the S-T interval, a change in the slope of the S-T interval, a change in one or both of the amplitude and width of the T-wave, and a change in one or both of the amplitude and width of the QRS complex. For different patients and different episodes for the patients, an ECG may manifest different changes. Correlating sensed cardiac signals with a template cardiac signal provides for detection of AMI even though particular morphological change may vary.

### Additional Notes

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a tangible computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus comprising:
an ambulatory cardiac signal sensing circuit configured to provide an electrical cardiac signal representative of cardiac activity of a subject;
a processor communicatively coupled to the ambulatory cardiac signal sensing circuit and including:
a feature module configured to:
identify a fiducial feature in the cardiac signal; and
locate one or more cardiac features in the cardiac signal using the fiducial feature; and
a correlation module configured to calculate a measure of similarity of morphology for a segment of the cardiac signal that includes the cardiac features; and
an ischemia detection module configured to:
detect a change in the measure of similarity; and
determine whether the detected change in the measure of similarity is indicative of ischemia and provide an indication of ischemia to a user or process according to the detected change in the measure of similarity.

2. The apparatus of claim 1,
wherein the ambulatory cardiac signal sensing circuit includes a first sensing channel configured to provide a first cardiac signal, and a second sensing channel configured to provide a second sensing channel, and
wherein the feature module is configured to:
identify the fiducial feature using the first cardiac signal; and
locate the cardiac features using the second cardiac signal, and
wherein the correlation module is configured to calculate the measure of similarity using a segment of the second cardiac signal.

3. The apparatus of claim 1,
wherein the feature module is configured to locate the cardiac features in the cardiac signal using SPs established in a template segment, wherein each SP corresponds to a turn encountered in the template segment, and
wherein the correlation module is configured to calculate a correlation coefficient (CC) that indicates a degree of similarity between a shape of the segment of the cardiac signal that includes the located SP features and a shape of the template segment.

4. The apparatus of claim 1,
wherein the feature module is configured to establish significant points (SPs) in a segment of the cardiac signal, wherein each SP corresponds to a turn encountered in the cardiac signal,
wherein the correlation module is configured to calculate a measure of similarity between the established SPs of the cardiac signal segment and the established SPs of the template signal, and
wherein the ischemia detection module is configured to detect a change in the measure of similarity of the established SPs.

5. The apparatus of claim 4, wherein the correlation module is configured to determine the measure of similarity using at least one of:
a number of SPs established for the cardiac signal segment;
a location of a turn corresponding to an SP of the cardiac signal segment;
a degree of turn of the cardiac signal segment corresponding to an SP; and
an amplitude of the cardiac signal segment at a corresponding turn to an SP.

6. The apparatus of claim 1, wherein the feature module is configured to locate one or more of:
a maximum value of the cardiac signal;
a minimum value of the cardiac signal;
a maximum slope of the cardiac signal;
a peak amplitude of a T-wave in the cardiac signal;
an end of a T-wave in the cardiac signal;
an S-wave to T-wave segment in the cardiac signal; and
a significant point in the cardiac signal.

7. The apparatus of claim 1,
wherein the correlation module is configured to:
align the fiducial feature of the of the cardiac signal segment with a corresponding feature in a template segment of the cardiac signal; and
calculate a correlation coefficient (CC) that indicates a degree of similarity between a shape of the segment of the cardiac signal that includes the located cardiac features and a shape of the template segment, and
wherein the ischemia detection module is configured to detect a change in the CC for the segment of the cardiac signal and determine whether the change is indicative of ischemia.

8. The apparatus of claim 7, including:
a therapy circuit communicatively coupled to the processor and configured to provide electrical pacing therapy to the subject,
wherein the ambulatory cardiac signal sensing circuit is configured to provide an electrical cardiac signal representative of cardiac depolarization, and
wherein the correlation module is configured to:
calculate the CC using a first template segment when the cardiac depolarization is representative of an intrinsic beat; and
calculate the CC using a second template segment when the cardiac depolarization is representative of a paced beat.

9. The apparatus of claim 1,
wherein the ambulatory cardiac signal sensing circuit is configured to sense a cardiac signal that includes a first cardiac signal segment and a second cardiac signal segment,
wherein the feature module is configured to identify a fiducial feature in the first cardiac signal segment and in the second cardiac signal segment, and
wherein the correlation module is configured to calculate a first measure of similarity for the first signal segment and calculate a second measure of similarity for the second segment.

10. The apparatus of claim 9,
wherein the first cardiac signal segment includes depolarization and the second cardiac segment include repolarization,
wherein the feature module is configured to identify the depolarization and repolarization, and
wherein the correlation module is configured to calculate a first measure of similarity for the first signal segment to a first template that includes depolarization and calculate a second measure of similarity for the second signal segment to a second template that includes repolarization.

11. The apparatus of claim 1, wherein the ischemia detection module is configured to:
calculate variation in the measure of similarity; and
deem that the change is indicative of ischemia when the calculated variation in the measure of similarity exceeds a specified threshold variation value.

12. A method comprising:
sensing at least one cardiac signal representative of cardiac activity of a subject using an ambulatory medical device (IMD);
identifying a fiducial feature in the cardiac signal;
locating one or more cardiac features in the cardiac signal using the fiducial feature;
calculating a measure of similarity of morphology of a segment of the cardiac signal that includes the located cardiac features;
detecting a change in the calculated measure of similarity; and
determining whether the detected change in the calculated measure of similarity is indicative of ischemia and providing an indication of ischemia to a user or process according to the detected change.

13. The method of claim 12,
wherein sensing at least one cardiac signal includes sensing a plurality of cardiac signals,
wherein a first cardiac signal is sensed using a first sensing channel and a second cardiac signal is sensed using a second sensing channel, and
wherein the fiducial feature is identified using the first cardiac signal, the cardiac features are located in the second cardiac signal, and the measure of similarity is calculated using a segment of the second cardiac signal.

14. The method of claim 12,
wherein locating cardiac features includes establishing significant points (SPs) in a segment of the cardiac signal, wherein each SP corresponds to a turn encountered in the cardiac signal,
wherein calculating a measure of similarity of morphology includes determining similarity between the SPs in the cardiac signal segment and SPs in a template of the signal segment, and
wherein detecting a change in the measure of similarity includes detecting a change in the established SPs.

15. The method of claim 12,
wherein locating cardiac features includes establishing significant points (SPs) in a segment of the cardiac signal, wherein each SP corresponds to a turn encountered in the cardiac signal, and
wherein calculating a measure of similarity of morphology includes calculating a correlation coefficient (CC) that indicates a degree of similarity between a shape of a segment of the cardiac signal that includes the established SPs and a shape of the template segment.

16. The method of claim 12, wherein locating cardiac features in the cardiac signal includes locating one or more of:
a maximum value of the cardiac signal;
a minimum value of the cardiac signal;
a maximum slope of the cardiac signal;
a peak amplitude of a T-wave in the cardiac signal;
an end of a T-wave in the cardiac signal;
an S-wave to T-wave segment in the cardiac signal; and
a significant point in the cardiac signal.

17. The method of claim 12, wherein calculating a measure of similarity of morphology includes:
aligning the fiducial feature of the of the cardiac signal segment with a corresponding feature in a template segment of the cardiac signal; and
calculating a correlation coefficient (CC) that indicates a degree of similarity between a shape of the segment of the cardiac signal that includes the feature and a shape of the template segment, and
wherein detecting a change in the calculated measure of similarity includes detecting a change in the CC that exceeds a CC threshold change value.

18. The method of claim 17, wherein calculating the CC includes:
determining at least one of patient heart rate and depolarization interval;
selecting a template segment from a plurality of template segments according to the determined rate or interval, wherein the plurality of template segments correspond to different ranges of rate or interval; and
determining the CC using the selected template segment.

19. The method of claim 12, wherein determining whether the change in the calculated measure of similarity is indicative of ischemia includes:
calculating a central tendency of the calculated measure of similarity of morphology; and
deeming that the change is indicative of ischemia when the calculated central tendency satisfies a specified threshold central tendency value.

20. The method of claim 12, wherein determining whether the change in the calculated measure of similarity is indicative of ischemia includes:
trending the change in the calculated measure of similarity of morphology; and
deeming whether the change is indicative of ischemia using the trended calculated measure of similarity.
